(19) United States
(12) Patent Application Publication
(10) Pub. No.: US 2008/0119731 A1
(43) Pub. Date: May 22, 2008

(54) PORTABLE ULTRASOUND WITH TOUCH SCREEN INTERFACE

(75) Inventors: Carlos Becerra, Atlanta, GA (US); William K. Chandler, Lawrenceville, GA (US); Abebaw Zeleke, Marietta, GA (US)

Correspondence Address: BRYAN W. BOCKHOP, ESQ.;BOCKHOP & ASSOCIATES, LLC, 2375 MOSSY BRANCH DR., SNELLVILLE, GA, 30078, US

(73) Assignee: NORTH AMERICAN MEDICAL CORPORATION, Marietta, GA (US)

(21) Appl. No.: 11/943,151

(22) Filed: Nov. 20, 2007

Related U.S. Application Data
(60) Provisional application No. 60/866,470, filed on Nov. 20, 2006.

(51) Int. Cl. A61B 8/00

(52) U.S. Cl. 600/437

(57) ABSTRACT

An ultrasound system for imaging an object using data received from an ultrasound transducer employs a first touch screen configured to display ultrasound images and to receive control inputs relating to the images. A second touch screen is configured as a user input device. A processor is coupled to the ultrasound transducer so as to receive data therefrom. The processor is also coupled to the first touch screen and to the second touch screen. The processor is configured to generate ultrasound images based on the data received from the ultrasound transducer. The processor is also configured to cause the first touch screen to display the ultrasound images. The processor is also configured to receive control input data from the first touch screen and the second touch screen.

### CROSS-REFERENCE TO RELATED APPLICATION

This application claims the benefit of U.S. Provisional Patent Application Ser. No. 60/866470, filed 11/20/2006, the entirety of which is hereby incorporated herein by reference.

### BACKGROUND OF THE INVENTION

1. Field of the Invention

The present invention relates to ultrasound systems and, more specifically, to a portable ultrasound system.

2. Description of the Prior Art

Ultrasound imaging has long been used by physicians, and other health care professionals, to view images of phenomena within a body. A typical ultrasound imaging system includes a transducer, a keyboard input, a computer and a display screen. The health care professional enters system input to the computer via the keyboard and views images on the display screen.

Typically, gels and other fluids get on the hands of the health care professional using an ultrasound imaging system. These fluids can be transferred to the keyboard. Thus, the keyboard must be cleaned regularly. However, this is a difficult process since the keyboard typically has an uneven surface.

One approach to facilitate cleaning of the keyboard is to place a plastic overlay over the keyboard. The overlay has a shape that is complimentary to the keys of the keyboard and prevents fluids from traveling into the keyboard. However, this approach still requires cleaning of the overlay, which may be difficult because of its irregular shape.

Several existing system have employed touch screen technology to provide a partial data input interface. However, such systems typically also include a traditional keyboard for data input or are too small for the effective display of ultrasound images.

Many clinicians need to perform ultrasound imaging away from a fixed site. Typical systems that employ keyboards are bulky, which makes performing ultrasound imaging away from a clinic difficult. One portable system includes a handheld personal digital assistant coupled to an ultrasound probe. This system has a relatively small screen that does not facilitate highly precise real-time diagnostics in many situations.

Therefore, there is a need for a portable ultrasound imaging system that has an easy to clean user interface.

### SUMMARY OF THE INVENTION

The disadvantages of the prior art are overcome by the present invention which, in one aspect, is an ultrasound system for imaging an object using data received from an ultrasound transducer. A first touch screen is configured to display ultrasound images and to receive control inputs relating to the images. A second touch screen is configured as a user input device. A processor is coupled to the ultrasound transducer so as to receive data therefrom. The processor is also coupled to the first touch screen and to the second touch screen. The processor is configured to generate ultrasound images based on the data received from the ultrasound transducer. The processor is also configured to cause the first touch screen to display the ultrasound images. The processor is also configured to receive control input data from the first touch screen and the second touch screen.

In another aspect, the invention is an ultrasound system that includes an ultrasound transducer, a first touch screen, a second touch screen, and a processor. The processor is configured to drive the ultrasound transducer and to receive signals therefrom. The processor is electrically coupled to the first touch screen and to the second touch screen. The processor includes a memory on which is stored a program. The program is configured to: drive the first touch screen so as to display on the first touch screen an ultrasound image corresponding to ultrasound data received from the ultrasound transducer, drive the second touch screen so as to display an image of a control interface that includes at least one specific input field indicative of a control value, receive at least one user input juxtaposed with the specific input field, and modify an operating parameter of the ultrasound system so as to conform to the control value. A battery is electrically coupled to, and configured for providing power to, the ultrasound transducer, the processor, the first touch screen and the second touch screen. A briefcase includes a first half that is hingedly attached to a second half. The first touch screen is mounted in the first half and the second touch screen is mounted in the second half. The processor is mounted in a selected one of the first half and the second half and the battery is mounted in a selected one of the first half and the second half.

In yet another aspect, the invention is an ultrasound apparatus for allowing a user to perform ultrasound imaging. The ultrasound apparatus includes an ultrasound transducer, a processor and a touch screen. The processor is configured to drive the ultrasound transducer and to receive signals therefrom. The touch screen is coupled to the processor. The touch screen is configured to display ultrasound images and to receive control inputs from the user so that the apparatus employs only the touch screen for both displaying the ultrasound images and for receiving the control inputs.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of an embodiment of an ultrasound system including a touch screen.

FIG. 2A is a photograph of a two touch screen embodiment.

FIG. 2B is a cross-sectional schematic diagram of the embodiment shown in FIG. 2A.

FIG. 3A is a schematic diagram of a two touch screen embodiment with a first touch screen configured as a display and a second touch screen configured as a button-type control interface.

FIG. 3B is a schematic diagram of a two touch screen embodiment with a first touch screen configured as a display and a second touch screen configured as a keyboard-type control interface.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

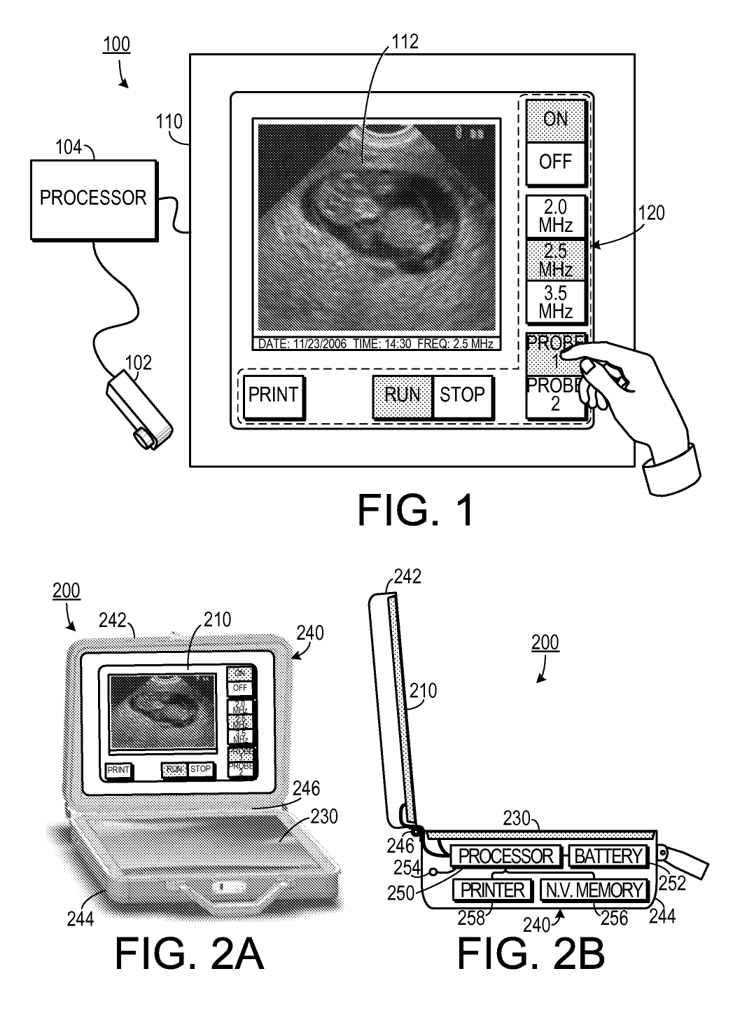

As shown in FIG. 1, one embodiment of the invention includes an ultrasound system 100 for imaging an object. The system 100 includes an ultrasound transducer 102 that generates an ultrasound imaging signal and that receives a return ultrasound imaging data signal (which could be in the form of analog data, digital data, or a combination thereof).

A processor 104 drives the ultrasound transducer 102 and receives a data signal therefrom. A touch screen 110 is coupled to the processor 104. The touch screen 110 is configured to display an ultrasound control interface120, which can take the form of images of control buttons, and is configured to receive control input from a user. The processor 104 is configured to generate an ultrasound image 112 of an object based on data received from the ultrasound transducer 102 and then display the image 112 of the object on the touch screen 110. The touch screen 110 presents the sole control interface to the user so that a keyboard is not necessary. Also, the touch screen is of sufficient size that a user is able to make a competent interpretation of the image 112 and so that a user is able to activate individual control buttons 120.

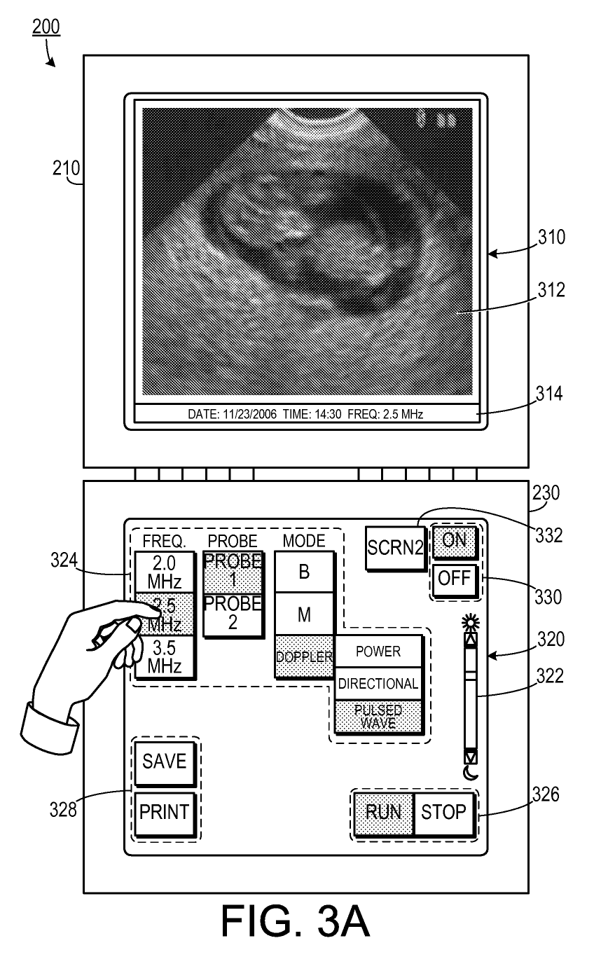

As shown in FIGS. 2A and 2B, one embodiment of an ultrasound system 200 includes a first touch screen 210 mounted in a first half 242 of a briefcase 240 and a second touch screen 230 mounted in a second half 244 of the briefcase 240. The first half 242 is coupled to the second half 244 by means of a hinge 246, or other similar connector. The briefcase 240 could include a rigid material, such as aluminum or plastic. Also mounted in a selected one of the top half 242 and the bottom half 244 is the processor 250 and a power supply (such as a battery 252), which supplies power to the processor 250, the first touch screen 210, the second touch screen 230 and the ultrasound transducer 102. While a battery power supply is shown, the ultrasound system 200 could also be configured to operate from an electrical outlet. A transducer port 254 facilitates coupling the ultrasound transducer 102 to the processor 250. A non-volatile memory 256 (such as a hard drive or a memory stick, etc.) is coupled to the processor 250 for storing the operating system, programming and information, such as ultrasound images. A printer 258 (e.g., a thermal printer) can be included with the system to facilitate printing of ultrasound images, or a printer port (not shown) can be coupled to the processor 250 to allow an external printer to be used.

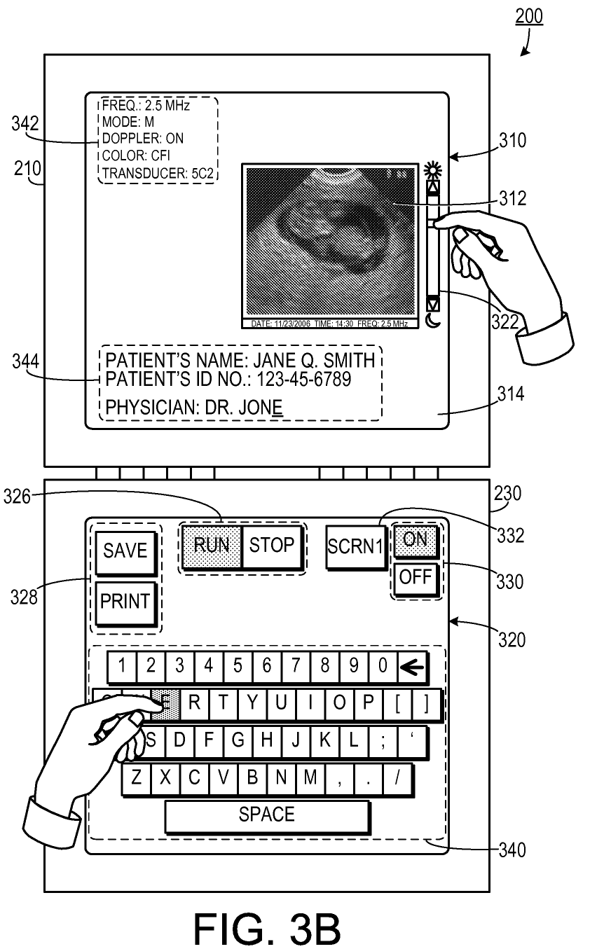

The touch screens 210 and 230 may be configured in several different ways according to the user's preferences. For example, as shown in FIG. 3A, the first touch screen 210 can be configured to display images 310, such as an ultrasound image 312 based on data received from the ultrasound transducer 102 and information 314 about the image. The second touch screen 230 can be configured as a control interface 320. The control interface 320 could include control field images and spatially juxtaposed data input fields. (Each combination of a control field image and a corresponding input field is referred to herein as a “button.”)

The buttons allow the user modify an operating parameters of the ultrasound system so as to conform to the control value displayed by the button. The control interface could include such buttons as: a brightness sliding scale control button 322, which controls the brightness of the display; on-off buttons 330, which can change cause the system to enter and exit a hibernate mode; run-stop buttons 326, which are used to begin and end an ultrasound testing episode; save-print buttons 328, which can cause the system to save information, such as a currently-displayed ultrasound image, on the non-volatile storage device and print such information. Printing an image can be effected by an internal printer or an external printer connected to the system. A plurality of process control buttons 324 allows the user to select the operating parameters of the system. A screen-select button 332 allows the user to select from one or more alternate screens (one of which will be shown in FIG. 3B).

An alternate display configuration is shown in FIG. 3B, in which the ultrasound image 312 is scaled down to allow information, such as operating information 342 and identifying information 344 to be displayed thereon. Scaling of the ultrasound image 312 can be effected by the user touching a corner of the ultrasound image 312 and dragging the corner to a location corresponding to the desired size of the ultrasound image 312. The brightness sliding scale button 322 is also moved up to the first touch screen 310. The second touch screen 230 can be configured as a keyboard 340 so as to allow the user to input text and numerical information.

The processor 250 is programmed to receive control inputs from the touch screens 210 and 230, to drive and receive ultrasound data from the ultrasound transducer 102 and to generate images for display on the touch screens 210 and 230. (As used herein “processor” includes all of the interfacing electronics necessary for the ultrasound system 200 to operate with the touch screens 210 and 230, the ultrasound transducer 102, the printer 258 and any other peripheral devices that may be used with the ultrasound system 200.) The processor 258 is programmed using one of the many programming tools available for touch screen systems.

In one commercial embodiment, a dual touch screen system can be purchased from Terason Ultrasound, Division of Teratech Corporation, 77 Terrace Hall Ave., Burlington, Mass. 01803. The system may run ultrasound software developed by Terason.

In one commercial embodiment, the ultrasound system 200 can include 256 beam-forming channels to create high image quality. 15-inch screens and a wide viewing angle provide a high level of visibility. Compact design of the system allows for diagnostic capability in cramped spaces, such as ambulances, emergency, and sports medicine clinics. The processor can use a Windows® platform and be programmed for auto-recognition of preset exams. The following are illustrative specifications for a commercial embodiment:

Physical Characteristics:
Dual 15″ XGA Color TFT Active Matrix Display (1024×768 per screen)
Dual Resistive Touch Screens
Intel 2.00 GHz Dual Core Processor
80 GB Hard Disk Drive
2 GB Memory
Onboard Intel Video Graphics (Shared Memory)
2-USB 2.0 Ports
Integrated Audio Controller
802.11 b/g Wireless Adapter
Windows XP Professional
Internal 8 Cell Battery
Built in AC Adapter
Weight: 9 lbs
Dimensions: 12″×14″×3.5″
Full feature ACPI power management, Stand-by, Suspend to Disk, and Suspend to RAM

Imaging Modes:
B-Mode
M-Mode
Power Doppler
Color Flow Imaging
Directional Power Doppler
Pulsed Wave Doppler

Transducers
4V2 (Vector)
Presets: Cardiac, Gynecological, Obstetrical, Pelvic, Prostate, Renal
5C2 (Curved Array)
Presets: Fetal Cardial, Gynecological, Obstectrical, Pelvic, Prostate, Renal
7L3 (Linear)
Presets: Breast, Carotid, Dialysis Access, Musculoskeletal, Testes, Thyroid, Vascular
Access, Venous
12L5 (Linear)
Breast, Carotid, Dialysis Access, Musculoskeletal, Testes, Thyroid, Vascular Access,
Venous

User Interface Features:
Pre-Programmed Hot Keys
Adjustable brightness
Adjustable color scheme
Controls: zoom, full screen, depth, size, gain, play/pause/next/back, invert, freeze, colorization, smoothing, persistence, MAP, sweep speed, time series image height, contrast, ellipse measurement, linear measurement, text, print, print preview, save as ULT, BMP, JPEG, TIF, export, new patient, patient info, image sorter
Image Display Properties: body marker, depth ruler, frame rate, measurement value, orientation logo, TGC Display
Needle Guide Display: Guide Lines, needle guide target indicator, biplanar grid selection
Annotation: Body Marker, Text
Help: Help Topics, Technical Support, About

System Parameters:
Static Image and Loop Image Storage
Storage capability of greater than 50,000 images
Up to 200 frames per second
256 Digital Beamforming Channels (128 Transmit, 128 Receive)

Because the input interface for the ultrasound system is a touch screen, cleaning of the interface is quite easy—requiring only a wiping off of the touch screen.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

1. An ultrasound system for imaging an object using data received from an ultrasound transducer, comprising:
a. a first touch screen that is configured to display ultrasound images and to receive control inputs relating to the images; and
b. a second touch screen that is configured as a user input device; and
c. a processor, coupled to the ultrasound transducer so as to receive data therefrom, and coupled to the first touch screen and to the second touch screen, the processor configured to generate ultrasound images based on the data received from the ultrasound transducer, the processor also configured to cause the first touch screen to display the ultrasound images, the processor also configured to receive control input data from the first touch screen and the second touch screen.

2. The ultrasound system of claim 1, wherein the first touch screen is hingedly coupled to the second touch screen.

3. The ultrasound system of claim 2, wherein the first touch screen is mounted in a first half of a briefcase and wherein the second touch screen is mounted in a second half of the briefcase.

4. The ultrasound system of claim 3, wherein the processor is mounted in a selected one of the first half of the briefcase and the second half of the briefcase.

5. The ultrasound system of claim 3, wherein a battery, electrically coupled to the first touch screen and to the second touch screen, is mounted in a selected one of the first half of the briefcase and the second half of the briefcase.

6. The ultrasound system of claim 1, wherein the processor is programmed to drive the first touch screen so as to receive a touch input indicative of a user's desire to change at least one dimension of an ultrasound image being displayed thereon and wherein the processor is programmed to drive the first touch screen so as to change the ultrasound image so as to have the dimension desired by the user.

7. The ultrasound system of claim 1, wherein the processor is programmed to:
a. drive the second touch screen so as to display an image of a control interface that includes at least one specific input field indicative of a control value;
b. receive at least one user input juxtaposed with the specific input field; and
c. modify an operating parameter of the ultrasound system so as to conform to the control value.

8. The ultrasound system of claim 7, wherein the operating parameter indicates a manner in which data is output onto a selected one of the first touch screen and the second touch screen.

9. The ultrasound system of claim 7, wherein the operating parameter indicates a control value used to drive the ultrasound probe.

10. The ultrasound system of claim 7, wherein the operating parameter indicates a manner in which data from ultrasound probe is processed.

11. The ultrasound system of claim 1, further comprising a nonvolatile storage device configured to store a plurality of ultrasound images.

12. The ultrasound system of claim 11, wherein the nonvolatile storage device comprises a hard drive.

13. An ultrasound system, comprising:
a. an ultrasound transducer;
b. a first touch screen;
c. a second touch screen;
d. a processor configured to drive the ultrasound transducer and to receive signals therefrom, the processor being electrically coupled to the first touch screen and to the second touch screen, the processor including a memory on which is stored a program that is configured to:
i. drive the first touch screen so as to display on the first touch screen an ultrasound image corresponding to ultrasound data received from the ultrasound transducer;
ii. drive the second touch screen so as to display an image of a control interface that includes at least one specific input field indicative of a control value;
iii. receive at least one user input juxtaposed with the specific input field; and
iv. modify an operating parameter of the ultrasound system so as to conform to the control value;
e. a battery, electrically coupled to, and configured for providing power to, the ultrasound transducer, the processor, the first touch screen and the second touch screen; and
f. a briefcase including a first half that is hingedly attached to a second half, the first touch screen mounted in the first half, the second touch screen mounted in the second half, the processor mounted in a selected one of the first half and the second half and the battery mounted in a selected one of the first half and the second half.

14. The ultrasound system of claim 13, wherein the operating parameter indicates a manner in which data is output onto a selected one of the first touch screen and the second touch screen.

15. The ultrasound system of claim 13, wherein the operating parameter indicates a control value used to drive the ultrasound probe.

16. The ultrasound system of claim 13, wherein the operating parameter indicates a manner in which data from ultrasound probe is processed.

17. The ultrasound system of claim 13, further comprising a nonvolatile storage device configured to store a plurality of ultrasound images.

18. The ultrasound system of claim 13, wherein the nonvolatile storage device comprises a hard drive.

19. An ultrasound apparatus for allowing a user to perform ultrasound imaging, comprising:
a. an ultrasound transducer;
b. a processor configured to drive the ultrasound transducer and to receive signals therefrom;
c. a touch screen, coupled to the processor, that is configured to display ultrasound images and to receive control inputs from the user so that the apparatus employs only the touch screen for both displaying the ultrasound images and for receiving the control inputs.
